(19) United States
(12) Patent Application Publication
(10) Pub. No.: US 2009/0240033 A1
(43) Pub. Date: Sep. 24, 2009

(54) AFFINITY MATRIX LIBRARY AND ITS USE

(76) Inventor: Rongxiu LI, Shanghai (CN)

Correspondence Address: LAHIVE & COCKFIELD, LLP;FLOOR 30, SUITE 3000, ONE POST OFFICE SQUARE, BOSTON, MA, 02109, US

(21) Appl. No.: 12/402,272

(22) Filed: Mar. 11, 2009

Related U.S. Application Data
(60) Provisional application No. 61/069,094, filed on Mar. 11, 2008.

(51) Int. Cl. C07K 1/14; C40B 30/04; C40B 40/04

(52) U.S. Cl. 530/344; 506/9; 506/15; 530/412; 530/413

(57) ABSTRACT

The invention relates, at least in part, to an affinity matrix library and the construction and use thereof. The library may be used, for example, for the enrichment of low-abundance proteins and depletion of abundant proteins in the search for biologically important proteins. The present invention also relates to a synthetic affinity matrix library comprising one or more ligand compounds with groups selected from amino, sulfhydryl, hydroxyl, carbonyl, and/or active hydrogen. The ligand compound may be attached to a base matrix.

### RELATED APPLICATIONS

This application claims the benefit of U.S. Provisional Patent Application Ser. No. 61/069,094, entitled “Affinity Matrix Library and Its Use”, filed Mar. 11, 2008. The entire content of the above-referenced patent application is incorporated herein by reference.

### BACKGROUND OF THE INVENTION

Many proteins that are present at low concentration (referred to herein as low-abundance proteins) in the cells and body fluid of multicellular and unicellular organisms are often important to the function of these organisms. These proteins can be difficult to detect due to low concentrations, variable concentration ranges (e.g., a range over 10 orders of magnitude), and a masking effect by high abundance proteins. Furthermore, the majority of low-abundance proteins are not accessible by high-throughput approaches commonly applied in the field. Accordingly, there remains a need for methodologies directed towards isolating low-abundance proteins.

### SUMMARY OF THE INVENTION

Accordingly, provided herein is a general affinity ligand matrix library that can be used, for example, to isolate low abundance protein(s) that can have therapeutic properties. In one aspect, the present invention relates to a general affinity ligand matrix library which includes at least one ligand compound with an active group selected from the group including an amino group, a sulfhydryl group, a hydroxyl group, a carbonyl group, and an active hydrogen group. In one embodiment of this aspect, the general affinity ligand matrix library includes approximately 300-1000 ligands. In a preferred embodiment of this aspect the general affinity ligand matrix library includes approximately 700 ligands. In another preferred embodiment of this aspect, the general affinity ligand matrix library includes synthetic ligands.

In one embodiment of this aspect, the general affinity ligand matrix library comprises a base matrix. In another embodiment of this aspect, the base matrix is any compound or material, wherein the material is particulate or non particulate, soluble or insoluble, or porous or nonporous. In a further embodiment of this aspect, the base matrix comprises activated agarose, silica, cellulose, glass, toyopearl, hydroxyethylmethacrylate, polyacrylamide, styrenedivinylbenzene, Hyper D, or perfluorocarbons.

In another embodiment of this aspect, the general affinity ligand matrix library includes at least one ligand compound that is saturated or unsaturated, linear, branched, or ring alkyl compound containing from 1 to 10 carbon or hetero atoms. In a further embodiment of this aspect, the general affinity ligand matrix library includes at least one ligand compound comprising from 2 to 20 carbon atoms, 0-6 nitrogen atoms, 0-6 oxygen atoms, 0-3 sulfur atoms, and 0-3 phosphorous atoms. In yet another embodiment of this aspect, the general affinity ligand matrix library includes at least one ligand compound that is linear or ring, saturated or unsaturated, containing from 2 to 20 carbon atoms, 0-6 nitrogen atoms, 0-6 oxygen atoms, 0-3 sulfur atoms, or 0-3 phosphorous atoms.

In one embodiment of this aspect, the general affinity ligand matrix library includes at least one ligand compound that is an alkyl. In another embodiment of this aspect, the alkyl group is a butyl, cyclohexyl, ethyl, hexyl, methyl, octyl pentyl, or propyl, and one or more hydrogens on these groups is optionally substituted with one or more substituents.

In another embodiment of this aspect, the ligand compound of the general affinity ligand matrix library includes at least one or more hydrogens which are optionally replaced with one or more substituents independently selected from the group including acyloxy or acylamino groups, sulphonyl groups or halogen atoms, amino groups, carbamoyl groups, carboxylic acid groups, hydroxyl groups, phosphoric acid groups, sulphonic acid groups, sulphamoyl groups; including aryl groups such as benzimidazole, benzoxazole, benzthiazole, catechol, p-chlorophenol, o-cresol, m-cresol, p-cresol, hydroquinone, 2-hydroxy-3-naphthoic acid, indazole, naphthalene, 1-naphthol, 2-naphthol, 1-naphthol-4-sulphonic acid, 2-naphthol-6-sulphonic acid, resorcinol, indazole, naphthyl, phenyl, phenol, 1-phenylpyrazole ring, and wherein these rings are optionally substituted with one or more substituents independently selected from the group consisting of alkyl groups containing from 1 to 10 carbon atoms, alkoxy groups containing from 1 to 10 carbon atoms, acyloxy or acylamino groups containing from 1 to 10 carbon atoms, alkylsulphonyl groups containing from 1 to 10 carbon atoms or halogen atoms, amino groups, carbamoyl groups, carboxylic acid groups, hydroxyl groups, phosphoric acid groups, sulphonic acid groups, or sulphamoyl groups.

In a further embodiment of this aspect, the ligand compound of the general affinity ligand matrix library is benzimidazole, benzoxazole, benzthiazole, catechol, p-chlorophenol o-cresol, m-cresol, p-cresol, hydroquinone, 2-hydroxy-3-naphthoic acid, indazole, naphthalene, 1-naphthol, 2-naphthol, 1-naphthol-4-sulphonic acid, 2-naphthol-6-sulphonic acid, resorcinol, indazole, naphthyl, phenyl, phenol, or 1-phenylpyrazole ring, and wherein these rings are optionally substituted with one or more substituents. In another embodiment of this aspect, at least one of the ligand compounds is optionally substituted with one or more substituents independently selected from the group consisting of alkyl groups containing from 1 to 10 carbon atoms, alkoxy groups containing from 1 to 10 carbon atoms, acyloxy or acylamino groups containing from 1 to 10 carbon atoms, alkylsulphonyl groups containing from 1 to 10 carbon atoms or halogen atoms, amino groups, carbamoyl groups, carboxylic acid groups, hydroxyl groups, phosphoric acid groups, sulphonic acid groups, or sulphamoyl groups.

In one embodiment of this aspect, the general affinity ligand matrix library includes at least one ligand compound that comprises an amino group. In another embodiment of this aspect, at least one of the ligand compounds of the general affinity ligand matrix library is 4-acetoxybenzylamine, 4-acetylaminobenzylamine, 1-adamantanamine, 3-aminoacetophenone, 4-aminoacetophenone, 1-aminoanthraquinone, p-aminoacetanilide, 9-aminoacridine, 4-aminoazobenzene, o-aminoazotoluene, p-aminobenzamide, 4-aminobenzamidine, p-aminobenzenesulphonamide, 2-aminobenzimidazole, o-aminobenzoic acid, m-aminobenzoic acid, p-aminobenzoic acid, 2-aminobenzothiazole, 4-aminobiphenyl, 6-amino caproic acid, 2-amino-5-chlorobenzoxazole, 4-amino-2,6-dinitrotoluene, 6-aminoindazole, 5-amino isophthalic acid, 1-amino-2-naphthol, 1-amino-3-naphthol, 1-amino-4-naphthol, 1-amino-5-naphthol, 1-amino-6-naphthol, 1-amino-7-naphthol, 1-amino-8-naphthol, 2-amino-1-naphthol, 2-amino-3-naphthol, 2-amino-4-naphthol, 2-amino-5-naphthol, 2-amino-6-naphthol, 2-amino-7-naphthol, 2-amino-8-naphthol, 1-amino-2-naphthol-4-sulfonic acid, 5-amino-1-naphthol-3-sulphonic acid, 6-amino-1-naphthol-3-sulphonic acid, 7-amino-1-naphthol-3-sulphonic acid, 1,8-diamino-3,6-dioxactane, 4-amino salicylic acid, 2-amino terephthalic acid, 2-amino-3-nitrophenol, 4-amino-2-nitrophenol, 2-aminophenol, 3-aminophenol, 4-aminophenol, 5-amino-1-phenylpyrazole, 3-aminophthalhydrazide, 3-amino-1-propanol, 2-aminopyridine, 3-aminopyridine, 4-amine-salicylic acid, amylamine, aniline, aniline-2,5-disulphonic acid, m-anisidine, o-anisidine, p-anisidine, arginine, asparagine, aspartic acid, benzidine, benzylamine, n-benzylaniline, n-benzyl-2-phenylethylamine, n-benzyltyramine, n-tert-butylaniline, p-butylaniline, n-butyl-benzylamine, p-chloroaniline, cyclohexylamine, cysteine, dehydroabietylamine, 2,4-diaminoanisole, m-diamino benzene, 1,4-diaminobutane, 4,4-diaminodiphenylmethane, 1,6-diamino hexane, 2,4-diamino-6-methylphenol, 2,4-diaminotoluene, o-dianisidine, 2,3-dichloroaniline, 2,4-dichloroaniline, 2,5-dichloroaniline, 2,6-dichloroaniline, 3,3-dichlorobenzidine, diethanolamine, diisopropylamine, 2,4-dimethylaniline, 2,6-dimethylaniline, 3,5-dinitroaniline, 2,6-diethylaniline, 2,4-dimethylaniline, 2,4-dinitroaniline, 2,6-dinitroaniline, ethanolamine, p-ethoxyaniline, ethylamine, n-ethylaniline, ethylenediamine, 2-furfurylamine, glutamic acid, glutamine, hexylamine, histamine, histidine, 4-hydroxybenzylamine, n-(2-hydroxyethyl)-benzylamine, iminodiacetic acid, isobutylamine, isoleucine, isopropylamine, 4-isopropylaniline, n-isopropylaniline, leucine, lysine, metanilic acid, methionine, 4-methoxyaniline-2-sulphoric acid, 2-methoxy-5-methylaniline, n-methylaniline, 4-methylbenzylamine, 4-methyl-3-nitroaniline, 3-methoxypropylamine, methyl 2-aminobenzoate, 4-methoxybenzylamine, 4-methylaniline-2-sulphonic acid, n-methylbenzylamine, 4,4-methylene-bis(2-chloroaniline), 4,4-methylene-bis(2-methylaniline), n-methylmetanilic acid, 2-methyl-3-nitroaniline, 2-methyl-4-nitro aniline, 2-methyl-5-nitroaniline, 2-methyl-6-nitroaniline, 3-methyl-o-phenylenediamine, 4-methyl-m-phenylenediamine, 4-methyl-o-phenylenediamine, 1-naphthylamine, 2-naphthylamine, 2-nitroaniline, 3-nitroaniline, 4-nitroaniline, octylamine, orthanilic acid, 4,4-oxydianiline 2-phenethylamine, phenylalanine, 1,4-phenylenediamine, 4-phthalazinedione, proline, propylamine, serine, sulphanilic acid, 4,4-thiodianiline, threonine, o-toluidine, p-toluidine, m-toluidine, 2,4,6-trichloroaniline, tryptamine, trptophan, tyramine, tyromine, or valine.

In one embodiment of this aspect, the general affinity ligand matrix library includes at least one ligand compound attached to the base matrix through activation and immobilization through amine reactive chemistry, sulfhydryl reactive chemistry, carbonyl chemistry, hydroxyl reactive chemistry, active hydrogen reactive chemistry, or photoreactive reactive chemistry. In another embodiment, the sulflhydryl reactive chemistry utilizes cyanogen bromide, carbonyl diimidazole, divinylsulfone, azlactone, cyanuric chloride, 2-fluoro-1-methylpyridinium toluene-4-sulfonate, tosyl chloride, tresyl chloride, iodoacetyl, bromoacetyl, maleimide, pyridyl disulfide, epoxyl, bisoxirane, or 5-thio-2-nitro benzoic acid as a reactant. In a further embodiment, the carbonyl chemistry utilizes hydrazide or reductive animation. In another embodiment, the hydroxyl reactive chemistry utilizes cyanogen bromide, cyanuric chloride, epoxyl, or bisoxirane. In yet another embodiment, the active hydrogen reactive chemistry utilizes a Diazonium or a Mannich Condensation. In an additional embodiment, the photoreactive reactive chemistry utilizes p-azidophenyl glyoxal, azidobenzoyl hydrazide, sulfosuccunimidyl-6-(4′-azido-2′-nitrophenylamino)hexanoate, or N-[4-(p-azidosalicylamido)butyl]-3′-(2′-pyridyldithio) propionamide.

In one aspect, the present invention relates to a method for using a general affinity ligand matrix library for the enrichment of low abundance proteins from a biological sample, wherein said method includes: (a) incubating a sample of interest with the general affinity ligand matrix library; and (b) separating the low abundance protein(s) from the high abundance proteins, wherein said low abundance protein(s) are enriched in the sample of interest.

In another aspect, the present invention relates to a method for using a general affinity ligand matrix library for the depletion of high abundance proteins from a biological sample, wherein said method includes: (a) incubating a sample of interest with the general affinity ligand matrix library; and (b) separating the high abundance protein(s) from the low abundance protein(s), wherein said high abundance proteins are depleted from the sample of interest.

In one embodiment of these aspects, the biological sample is body fluid selected from the group consisting of ascites, blood, plasma, serum, chyle, semen, interstitial fluid, lymph fluid, menses, breast milk, sweat, tears, urine, vaginal lubrication, Cowper's fluid or pre-ejaculatory fluid, female ejaculate, mucus, pleural fluid, pus, saliva, sebum (skin oil), chyme, and vomit. In another embodiment of these aspects, the biological sample is of animal origin. In yet another embodiment of these aspects, the biological sample is from mice, rats, pigs, guinea pigs, rabbits, horses, cows, dogs, cats, humans, or monkeys.

In one embodiment of these aspects, at least one ligand of said general affinity ligand matrix library comprises a residue of aniline and/or 1,8-diamino-3,6-dioxactane. In another embodiment of these aspects, at least one ligand of said general affinity ligand matrix library comprises a residue of 1,6-diamino hexane and/or hexylamine. In another embodiment of these aspects, at least one ligand of said general affinity ligand matrix library comprises a residue of m-amino phenol and/or m-diamino benzene. In a further embodiment of these aspects, at least one ligand of said general affinity ligand matrix library comprises a residue of 4-aminobenzamidine and/or L-proline. In another embodiment of these aspects, at least one ligand of said general affinity ligand matrix library comprises a residue of 1,6-diamino hexane. In yet another embodiment of these aspects, at least one ligand of said general affinity ligand matrix library comprises a residue of aniline and/or butylamine. In a further embodiment of these aspects, at least one ligand of said general affinity ligand matrix library comprises a residue of 3-aminophthalhydrazide and/or L-phenylalanine. In an additional embodiment of these aspects, at least one ligand of said general affinity ligand matrix library comprises a residue of dehydroabietylamine and/or 1,6-diamino hexane. In another embodiment of these aspects, at least one ligand of said general affinity ligand matrix library comprises a residue of dehydroabietylamine and/or cyclohexylamine. In another embodiment of theses aspects, at least one ligand of said general affinity ligand matrix library comprises a residue of 3-picolylamine and residue of octylamine. In yet another embodiment of these aspects, at least one ligand of said general affinity ligand matrix library comprises a residue of 3-picolylamine and/or residue of cyclohexylamine. In a further embodiment of these aspects, at least one ligand of said general affinity ligand matrix library comprises a residue of 3-picolylamine and/or 4-aminobenzamidine. In an additional embodiment of these aspects, at least one ligand of said general affinity ligand matrix library comprises a residue of 3-picolylamine and/or 2-aminoterephthalic acid. In another embodiment, at least one ligand of said general affinity ligand matrix library comprises a residue of 1-adamantanamine and/or 4-aminobenzamidine. In another embodiment, at least one ligand of said general affinity ligand matrix library comprises a residue of 4-amine-salicylic acid and/or aniline. In yet another embodiment, at least one ligand of said general affinity ligand matrix library comprises a residue of iminodiacetic acid and/or 4-aminobenzamidine. In a further embodiment, at least one ligand of said general affinity ligand matrix library comprises a residue of lysine and/or butylamine. In another embodiment, at least one ligand of said general affinity ligand matrix library comprises a residue of lysine and/or aniline. In an additional embodiment, at least one ligand of said general affinity ligand matrix library comprises a residue of lysine and/or 1-aminoanthroquinone. In yet another embodiment of these aspects, at least one ligand of said general affinity ligand matrix library comprises a residue of 5-amine-isophthalic acid.

In one aspect, the present invention relates to a method for separating proteins or peptides from a mixture, which includes contacting the mixture with a general affinity ligand matrix library, such that a protein- or peptide-affinity ligand complex forms, and removing the protein- or peptide-affinity ligand complex from the mixture, such that proteins or peptides are separated from the mixture.

In one embodiment of these methods, the protein is an antibody, hormone, receptor, or a cell adhesion molecule. In another embodiment of these aspects, the mixture is plasma, blood, protein extract, protein lysate, a biological sample, or any proteinacious sample. In another embodiment of these aspects the general affinity ligand matrix library comprises a label. In a further embodiment of these aspects, the general affinity ligand matrix library comprises a ligand with an active group. In yet another embodiment of these aspects, the active group is an amino group, a sulfhydryl group, hydroxyl group, a carbonyl group, or an active hydrogen group. In an additional embodiment of these aspects, the affinity ligand matrix library is a general affinity ligand matrix library.

In one aspect, the present invention relates to a method of depleting one or more peptides or proteins from a mixture, including contacting the initial mixture with a chemically reactive matrix to form a protein or peptide complex with the chemically reactive matrix, removing the protein or peptide complex from the mixture, such that the proteins or peptides are depleted from the mixture.

In one embodiment, the present invention relates to a method of identifying a selectively reactive affinity ligand for a protein, which includes contacting the protein with a general affinity ligand matrix library, allowing one or more protein complexes to form with the selectively reactive affinity ligand, and identifying the protein complex, such that the selectively reactive affinity ligand for the protein is identified.

In another embodiment of these aspects, the protein is a protein of therapeutic use.

In one aspect, the present invention relates to a method of selectively obtaining a low-abundance protein from a sample, comprising contacting the sample with a general reactive affinity ligand such that a protein complex with the low-abundance protein and the selectively reactive affinity ligand is formed, and separating the protein complex from the mixture, such that the low-abundance protein is selectively obtained. In another embodiment of these aspects, the selectively reactive affinity ligand is identified using the method of these aspects and embodiments.

In one aspect, the present invention relates to a general affinity ligand matrix library, wherein the affinity matrix of each component of the library comprises the general formula: M-L-R1(R2).

In one embodiment of this aspect, M represents activated agarose, silica, cellulose, glass, toyopearl, hydroxyethylmethacrylate, polyacrylamide, styrenedivinylbenzene, Hyper D, perfluorocarbons, metal oxide, glass or silica matrices, optionally coated with an organic polymer. In another embodiment of this aspect, L represents a 1,1′-carbonyldiimidazole activated linkage, cyanuric bromide activated linkage, cyanuric chloride activated linkage, cyanuric floride activated linkage, cyanogen bromide activated linkage, epoxy activated linkage, 2-fluoro-1-methylpyridiniumtoluene-4-sulphonate activated linkage, glycidoxypropyltrimethoxysilane activated linkage, sodium meta-periodate activated linkage, sulphonylchloride activated linkage, tosyl activated linkage, tresyl activated linkage, 2,2,2-trifluoroethanesulphonyl chloride activated linkage, or vinylsulphone activated linkage. In a further embodiment of this aspect, L represents N-ethoxycarbonyl-2-ethoxy-1,2-dihydroquinoline, dicyclohexyl carbodiimide and 1-ethyl-3-(3-dimethylaminopropyl)-carbodiimide. In another embodiment of this aspect, R1(R2) represents the immobilization of one or two chemical groups, wherein the chemical groups are alkyl or aryl groups. In a further embodiment of this aspect, R1 and R2 are selected from the group comprising, 4-acetoxybenzylamine, 4-acetylaminobenzylamine, 1-adamantanamine, 3-aminoacetophenone, 4-aminoacetophenone, 1-aminoanthraquinone, p-aminoacetanilide, 9-aminoacridine, 4-aminoazobenzene, o-aminoazotoluene, p-aminobenzamide, 4-aminobenzamidine, p-aminobenzenesulphonamide, 2-aminobenzimidazole, o-aminobenzoic acid, m-aminobenzoic acid, p-aminobenzoic acid, 2-aminobenzothiazole, 4-aminobiphenyl, 6-amino caproic acid, 2-amino-5-chlorobenzoxazole, 4-amino-2,6-dinitrotoluene, 6-aminoindazole, 5-amino isophthalic acid, 1-amino-2-naphthol, 1-amino-3-naphthol, 1-amino-4-naphthol, 1-amino-5-naphthol, 1-amino-6-naphthol, 1-amino-7-naphthol, 1-amino-8-naphthol, 2-amino-1-naphthol, 2-amino-3-naphthol, 2-amino-4-naphthol, 2-amino-5-naphthol, 2-amino-6-naphthol, 2-amino-7-naphthol, 2-amino-8-naphthol, 1-amino-2-naphthol-4-sulfonic acid, 5-amino-1-naphthol-3-sulphonic acid, 6-amino-1-naphthol-3-sulphonic acid, 7-amino-1-naphthol-3-sulphonic acid, 1,8-diamino-3,6-dioxactane, 4-amino salicylic acid, 2-amino terephthalic acid, 2-amino-3-nitrophenol, 4-amino-2-nitrophenol, 2-aminophenol, 3-aminophenol, 4-aminophenol, 5-amino-1-phenylpyrazole, 3-aminophthalhydrazide, 3-amino-1-propanol, 2-aminopyridine, 3-aminopyridine, 4-amine-salicylic acid, amylamine, aniline, aniline-2,5-disulphonic acid, m-anisidine, o-anisidine, p-anisidine, arginine, asparagine, aspartic acid, benzidine, benzylamine, n-benzylaniline, n-benzyl-2-phenylethylamine, n-benzyltyramine, n-tert-butylaniline, p-butylaniline, n-butyl-benzylamine, p-chloroaniline, cyclohexylamine, cysteine, dehydroabietylamine, 2,4-diaminoanisole, m-diamino benzene, 1,4-diaminobutane, 4,4-diaminodiphenylmethane, 1,6-diamino hexane, 2,4-diamino-6-methylphenol, 2,4-diaminotoluene, o-dianisidine, 2,3-dichloroaniline, 2,4-dichloroaniline, 2,5-dichloroaniline, 2,6-dichloroaniline, 3,3-dichlorobenzidine, diethanolamine, diisopropylamine, 2,4-dimethylaniline, 2,6-dimethylaniline, 3,5-dinitroaniline, 2,6-diethylaniline, 2,4-dimethylaniline, 2,4-dinitroaniline, 2,6-dinitroaniline, ethanolamine, p-ethoxyaniline, ethylamine, n-ethylaniline, ethylenediamine, 2-furfurylamine, glutamic acid, glutamine, hexylamine, histamine, histidine, 4-hydroxybenzylamine, n-(2-hydroxyethyl)-benzylamine, iminodiacetic acid, isobutylamine, isoleucine, isopropylamine, 4-isopropylaniline, n-isopropylaniline, leucine, lysine, metanilic acid, methionine, 4-methoxyaniline-2-sulphoric acid, 2-methoxy-5-methylaniline, n-methylaniline, 4-methylbenzylamine, 4-methyl-3-nitroaniline, 3-methoxypropylamine, methyl 2-aminobenzoate, 4-methoxybenzylamine, 4-methylaniline-2-sulphonic acid, n-methylbenzylamine, 4,4-methylene-bis(2-chloroaniline), 4,4-methylene-bis(2-methylaniline), n-methylmetanilic acid, 2-methyl-3-nitroaniline, 2-methyl-4-nitroaniline, 2-methyl-5-nitroaniline, 2-methyl-6-nitroaniline, 3-methyl-o-phenylenediamine, 4-methyl-m-phenylenediamine, 4-methyl-o-phenylenediamine, 1-naphthylamine, 2-naphthylamine, 2-nitroaniline, 3-nitroaniline, 4-nitroaniline, octylamine, orthanilic acid, 4,4-oxydianiline 2-phenethylamine, phenylalanine, 1,4-phenylenediamine, 4-phthalazinedione, proline, propylamine, serine, sulphanilic acid, 4,4-thiodianiline, threonine, o-toluidine, p-toluidine, m-toluidine, 2,4,6-trichloroaniline, tryptamine, trptophan, tyramine, tyromine, and valine. In yet another embodiment of this aspect, R1 and R2 represent thiols. In a further embodiment of this aspect, R1 and R2 are selected from the group comprising, ethylthiol, thio-p-cresol, thioglycolic acid, thiophenol. In an additional embodiment of this aspect, R1 and R2 are selected from the group of amines of Table 1.

In one aspect, the present invention relates to a general affinity ligand matrix library, wherein the affinity matrix of each component of the library comprises the general formula: M-L-R1(R2), and wherein M represents sepharose, L represents an epoxy activated linkage or a cyanuric chloride activated linkage, and R1 and R2 are selected from the group of amines of Table 1.

In another embodiment of these aspects, the present invention relates to a process for the enrichment of low abundance proteins from proteinacious samples comprising carrying out affinity chromatography using as the general affinity matrix of the present invention. In yet another embodiment of these aspects, the base matrix is optionally activated agarose, cellulose, silica or glass.

### BRIEF DESCRIPTION OF THE FIGURES

FIG. 1 depicts a synthetic pathway for generating affinity ligands. Sepharose 4B was activated with epichlorohydrin. Amino compounds were coupled with 1,4-dioxane.

FIGS. 2A-2D depict the SDS-PAGE anaylsis of a) enrichment, b) depletion, c) one-step purification, and d) one-step purification via flow-through effects of synthetic affinity ligand on leech proteins. Lane 1 is crude leech extract, Lane M is the protein marker. FIG. 2A: lanes 2-7 show the 0.1M acetic acid (HAC) eluates of columns A8-47, A29-32, A6-2, A4-11, A11-70, and A2, respectively. FIG. 2B: lane 2 is the flow-through of column A15, lane 3 depicts the 0.1M HAC eluate of column A15, lane 4 is the flow-through of column A6, and lane 5 depicts the 0.1M HAC eluate of column A6. FIG. 2C: lanes 2-5 are the 0.1M HAC eluates of columns A903, NX, A83-83, and A32-53, respectively. FIG. 2D: lane 2 is the flow-through of column A8-21, lane 3 depicts the 0.1M HAC eluate of column A8-21, lane 4 depicts the flow-through of column A8-27, and lane 5 is the 0.1M HAC eluate of column A8-27.

FIG. 3 depicts the SDS-PAGE analysis of the depletion of high abundance proteins from human plasma with column 10-2: Lanes from left to right are: protein markers, original sample, flow-through fraction and elution fraction.

FIG. 4 depicts the SDS-PAGE analysis of the enrichment of low abundance proteins from human plasma with column 13-9.

FIG. 5 depicts the SDS-PAGE analysis of the enrichment of low abundance proteins from human plasma with column 13-18.

FIG. 6 depicts the SDS-PAGE analysis of the enrichment of low abundance proteins from human plasma with column 16-8.

### DETAILED DESCRIPTION OF THE INVENTION

The separation and characterization of low-abundance proteins is an area of importance, especially with regard to the identification of disease biomarkers, for diagnosing a disease at the biochemical level, for clinical studies to assay the treatment effects of different drug therapies, and for discriminating the responses of different patients to the same medical treatment. Thus, methods for separating and characterizing low-abundance proteins are of great interest to the pharmaceutical industry.

The major obstacle for the enrichment of low-abundance proteins is the presence of many other proteins in the sample of interest. Multi-dimensional liquid chromatography treatment to divide a sample of interest into sub-fractionations is one of the strategies used; however, it is limited by the resolution and sample capacity of the technique. Most methods for the detection of low-abundance proteins and their expression patterns require a reduction of the protein sample complexity. Only focused methods, such as antibody-based approaches, make it possible to isolate and characterize low abundance species. Depletion of abundant proteins allows for detection of proteins that co-migrate with, and are masked by, high abundance proteins during 1-D or 2-D gel electrophoresis.

The affinity purification system provided herein relies on specific, reversible, and non-covalent interactions between synthetic ligands and target proteins. These non-covalent interactions can be classified as ionic interactions, van der Waals interactions, hydrogen bonds, and hydrophobic interactions. They are responsible for the three-dimensional (3D) configuration and flexibility of target proteins and synthetic ligands. When the synthetic ligand is in proximity to the target protein, ionic interactions (salt bridge), van der Waals interactions, hydrogen bonds, and hydrophobic interactions will occur. The molecular distance between ligand and protein, and the polarity and electronegativity of the ligand and protein will determine the intensity of these interactions and the affinity force between synthetic ligand and protein. A high affinity force between the synthetic ligand and protein will lead to a highly stable ligand-protein complex. Because the binding is highly specific, affinity purification decreases non-specific interactions and eliminates undesirable contaminants, thereby making it a promising technique to deplete undesirable protein, purify specific proteins in one-step, and to enrich low-abundance proteins.

In order that the present invention may be more readily understood, certain terms are first defined. Additional terms are set forth throughout the detailed description.

The term “general” includes the term “not specialized.”

The term “general affinity ligand matrix library” includes a library of affinity ligands that are not specifically targeted to a single protein. That is the “general affinity ligand matrix library” of the instant invention contains multiple, varying ligands (e.g., the ligands of Table 1) that therefore target different proteins in a proteinacious sample. The term “general affinity ligand matrix library” also includes a library of affinity ligands that is not limited by scope, area, or application; accordingly, the general affinity ligand matrix library may bind one or more proteins or peptides with different characteristics. For example, the general affinity ligand matrix library is not specific for one specific protein or peptide, or one particular type of protein or peptide (e.g. hydrophobic, hydrophilic, glycosylated, acidic, basic). Furthermore, the affinity ligands of the library are attached to a support matrix.

As used herein, the term “synthetic affinity ligand” refers to small molecule ligands that have been bonded to the support matrix via organic synthesis methodologies.

The term “matrix” includes the term “support matrix.” In one embodiment, the matrix is a chemically reactive matrix.

The term “low abundance proteins” includes proteins present at a low concentration in a protein sample of interest. For example, the concentration of the low abundance protein of interest may be less than 51% of the total protein concentration in the sample. Also, for example, the concentration of the low abundance protein of interest may be less than 50%, 40%, 30%, 25%, 20%, 15%, 10%, 5%, 1%, 0.1%, 0.01%, 0.001%, 0.0001%, 0.00001%, 0.000001%, or 0.0000001% of the total protein concentration in the sample.

In contrast, the term “high abundance proteins” includes proteins present at a high concentration in a protein sample of interest. For example, the concentration of the high abundance protein(s) of interest may be greater than 50%, 40%, 30%, 25%, 20%, 15%, 10%, 5%, 1%, 0.1%, 0.01%, 0.001%, 0.0001%, 0.00001%, 0.000001%, or 0.0000001% of the total protein concentration in the sample.

The term “protein sample of interest” includes protein lysates, protein extracts, ascites, blood, plasma, serum, urine, lymph, interstitial fluid, amniotic fluid, aqueous humour, cerumen (e.g., earwax), cowper's fluid, chyme, breast milk, feces, mucus (e.g., nasal drainage and phlegm), pleural fluid, pus, saliva, sebum (e.g., skin oil), semen, sweat, tears, vaginal secretion, vomit, any biological sample, or any proteinacious sample.

The term “subfraction” includes a quantity of a substance (e.g., protein or neucleic acid) collected from a sample of interest in a fractionating separation process. In such a process, a protein sample is separated into fractions, which have varying characteristics. Furthermore, the fractions may be further separated to achieve fractions of the original fractions and so forth. For example, subfractionation can be performed such that the low abundance proteins of interest no longer co-exist with high abundance proteins

As used herein, the term “ligand” includes a molecule that has the ability to bind to or associate with a protein or peptide.

The general formula “M-L-R1(R2)” represents the affinity ligand matrix of the present invention.

As used in the general formula M-L-R1(R2), “M” represents a support matrix, which can be any compound or material, wherein the compound or material can be particulate or non-particulate, soluble or insoluble, porous or non-porous, wherein the compound or material provides a convenient means of linkage point for affinity ligands. Insoluble support matrices such as a naturally occurring polymer, for example a polypeptide or protein such as cross linked albumin or a polysaccharide such as agarose, alginate, carrageenan, chitin, cellulose, dextran or starch, m hydrolysed starch; synthetic polymers such as polyacrylamide, polystyrene, polyacrolein, polyethylene glycol, polyvinyl alcohol, polymethylacrylate, perfluorocarbon; inorganic compounds such as silica, glass, kieselguhr, alumina, iron oxide or other metal oxides or co-polymers consisting of any combination of two or more of a naturally occurring polymer, synthetic polymer or inorganic compounds.

As used in the general formula M-L-R1(R2), “L” represents an oxygen atom, a sulphur atom, a —COO— group, a CONH group, an NHCO group, a N(H) group, a triazine ring, a covalent bond or other linker capable of linking R1(R2) with M, such as otherwise described herein;

As used in the general formula M-L-R1(R2), “R1(R2)”, represents the immobilization of one or two chemical groups, of alkyl or aryl groups containing from 1 to 10 carbon atoms, of homo- or hetero-, of linear or ring, of saturated or unsaturated, containing from 2 to 20 carbon atoms, 0-6 nitrogen atoms, 0-6 oxygen atoms, 0-3 sulfur atoms, and 0-3 phosphorous atoms, including alkyl groups (e.g., butyl, cyclohexyl, ethyl, hexyl, methyl, octyl, pentyl, propyl), where one or more hydrogens on these groups is optionally replaced with one or more substituents independently selected from the group consisting of acyloxy or acylamino groups, sulphonyl groups or halogen atoms, amino groups, carbamoyl groups, carboxylic acid groups, hydroxyl groups, phosphoric acid groups, sulphonic acid groups, sulphamoyl groups; including aryl groups such as benzimidazole, benzoxazole, benzthiazole, catechol, p-chlorophenol o-cresol, m-cresol, p-cresol, hydroquinone, 2-hydroxy-3-naphthoic acid, indazole, naphthalene, 1-naphthol, 2-naphthol, 1-naphthol-4-sulphonic acid, 2-naphthol-6-sulphonic acid, resorcinol, indazole, naphthyl, phenyl, phenol, 1-phenylpyrazole, wherein these rings can be optionally substituted with one or more substituents independently selected from the group consisting of alkyl groups containing from 1 to 10 carbon atoms, alkoxy groups containing from 1 to 10 carbon atoms, acyloxy or acylamino groups containing from 1 to 10 carbon atoms, alkylsulphonyl groups containing from 1 to 10 carbon atoms or halogen atoms, amino groups, carbamoyl groups, carboxylic acid groups, hydroxyl groups, phosphoric acid groups, sulphonic acid groups, or sulphamoyl groups.

In one embodiment, “R1(R2)” is referred to as the ligand compound.

The theory of library design is based on the “lock and key” model of protein and ligand interactions. Specific polypeptide chains of each protein may spontaneously fold into a unique 3D form in particular environments. Different proteins may have different conformations in the same buffer environment. The many unique cavities and grooves in the 3D confirmation of proteins are different among different proteins. If these cavities and grooves are considered the “lock” of protein-ligand interactions, the smaller ligand molecule whose confirmation would fit well in these cavities and grooves could be considered the “key.” Although molecular interactions are not completely ridged and protein-ligand interactions may be induced fit or allosteric, a special “lock” (the cavities and grooves of protein) always needs the special “key” (the affinity ligand) to fit. If the size, shape, and functionality of synthetic ligands are varied enough, proteins with unique grooves and cavities can be picked out using at least one best binding affinity ligand. Using this rationale a large library of “keys” (affinity ligands) has been constructed. Given that the 3D structure of most proteins is currently unknown, and the simulation of molecular docking and modeling relies on the known structure of proteins, the present invention, which relates to large scale screening of affinity ligands, is an improved method for protein purification and further study of proteins.

The present invention, in part, relates to a novel general affinity ligand matrix library preparation on a matrix which may consist of solid, semi-solid, particulate or colloidal materials, or soluble polymers. In one embodiment the novel general affinity ligand matrix library, is not specialized (e.g. the general affinity ligand matrix library contains multiple, varying ligands). The invention further relates to said novel general affinity ligand matrix library and the preparation and use thereof in the proteomic research and diagnostics for enrichment of low-abundance proteins and/or the depletion of high abundance proteins.

The present invention further relates to a synthetic affinity ligand chromatographic matrix library, the preparation and the use of the chromatographic matrix library in the enrichment of low abundance proteins and/or depletion of high abundance proteins in the field of proteomics of biological samples.

The current invention is based on the notion that each group specific ligand based chromatographic matrix can extract a subfraction of proteins from a biological sample. A library of chromatographic matrix could divide the proteins in a biological sample into many subfractions so that the low abundance proteins of interest no longer co-exist with high abundance proteins.

The affinity matrix of the present invention comprises a solid, usually permeable, support matrix, to which a ligand is covalently attached. The affinity matrix is packed in a chromatographic column. Target proteins in the sample are delayed in the support matrix under near physiological conditions when the sample is passed over and the column is washed with equilibration buffer. The delayed target proteins can be eluted in their pure form. When the ligand binds proteins with similar features, it is defined as a group specific ligand. The combination of group specific ligands and the affinity separation methods of the present invention allows for the separation of complex proteinacious samples into subfractions of reduced complexity.

A feature of the present invention is the provision of a general tool for the enrichment (i.e., increasing the abundance of a protein) of low abundance proteins and the depletion (i.e., decreasing the abundance of a protein) of high abundance proteins in the field of proteomics. The library of affinity matrices with ligands of different chemical structure provided herein, have the ability to interact with different groups of proteins. A particularly effective ligand structure for a given group of proteins is identified by assessment of the general affinity ligand matrix library provided by the invention. By way of example, a group of high abundance proteins could be bound onto affinity matrices and leave low abundance proteins in the flow through fraction; another example is a group of low abundance proteins could be bound onto and eluted from affinity matrices, with enrichment of more than 10-fold, eliminating the masking effect of high abundance proteins in the flow through fraction.

Accordingly, the present invention relates to affinity matrix with the general formula (a):

(M-L-R1(R2)) (a)

wherein, R1(R2), represents the immobilization of one or two chemical groups, alkyl or aryl groups containing from 1 to 10 carbon atoms, of homo- or hetero-, of linear or ring, of saturated or unsaturated, containing from 2 to 20 carbon atoms, 0-6 nitrogen atoms, 0-6 oxygen atoms, 0-3 sulfur atoms, and 0-3 phosphorous atoms, including alkyl groups (e.g., butyl, cyclohexyl, ethyl, hexyl, methyl, octyl, pentyl, propyl), where one or more hydrogens on these groups is optionally replaced with one or more substituents independently selected from the group consisting of acyloxy or acylamino groups, sulphonyl groups or halogen atoms, amino groups, carbamoyl groups, carboxylic acid groups, hydroxyl groups, phosphoric acid groups, sulphonic acid groups, sulphamoyl groups; including aryl groups such as benzimidazole, benzoxazole, benzthiazole, catechol, p-chlorophenol o-cresol, m-cresol, p-cresol, hydroquinone, 2-hydroxy-3-naphthoic acid, indazole, naphthalene, 1-naphthol, 2-naphthol, 1-naphthol-4-sulphonic acid, 2-naphthol-6-sulphonic acid, resorcinol, indazole, naphthyl, phenyl, phenol, 1-phenylpyrazole, wherein these rings can be optionally substituted with one or more substituents independently selected from the group consisting of alkyl groups containing from 1 to 10 carbon atoms, alkoxy groups containing from 1 to 10 carbon atoms, acyloxy or acylamino groups containing from 1 to 10 carbon atoms, alkylsulphonyl groups containing from 1 to 10 carbon atoms or halogen atoms, amino groups, carbamoyl groups, carboxylic acid groups, hydroxyl groups, phosphoric acid groups, sulphonic acid groups, or sulphamoyl groups.

L represents an oxygen atom, a sulphur atom, a —COO— group, a CONH group, a NHCO group, a N(H) group, a triazine ring, a covalent bond or other linker capable of linking R1(R2) with M, such as otherwise described herein; and

M represents a support matrix, which can be any compound or material, wherein the compound or material can be particulate or non-particulate, soluble or insoluble, porous or non-porous, wherein the compound or material provides a convenient means of linkage point for affinity ligands. Insoluble support matrices such as a naturally occurring polymer, for example a polypeptide or protein such as cross linked albumin or a polysaccharide such as agarose, alginate, carrageenan, chitin, cellulose, dextran or starch, m hydrolysed starch; synthetic polymers such as polyacrylamide, polystyrene, polyacrolein, polyethylene glycol, polyvinyl alcohol, polymethylacrylate, perfluorocarbon; inorganic compounds such as silica, glass, kieselguhr, alumina, iron oxide or other metal oxides or co-polymers consisting of any combination of two or more of a naturally occurring polymer, synthetic polymer or inorganic compounds.

The variable R1(R2) may also be referred to herein as the “ligand compound.”

The term “alkyl group containing from 1 to 10 carbon atoms” as used herein, alone or in combination, refers to a straight or branched or ring, saturated or unsaturated, homo- or hetero hydrocarbon chain having 1 to 10 carbon atoms such as, e.g., N-butyl, sec-butyl, tert-butyl, 2,2-dimethylpropyl ethyl, n-hexyl isobutyl, isopropyl, n-hexyl, 2-methylbutyl, 3-methylbutyl, 4-methylpentyl, neopentyl, n-pentyl, or n-propyl.

The term “alkoxy group containing from 1 to 10 carbon atoms” as used herein, alone or in combination, refers to a straight or branched or ring, saturated or unsaturated, homo- or hetero monovalent or divalent substituent comprising an alkyl group containing from 1 to 10 carbon atoms linked through an ether oxygen having its free valence bond from the ether oxygen and having 1 to 6 carbon atoms, e.g., butoxy, ethoxy, isopropoxy, methoxy, propoxy, or pentoxy.

The term “halogen” means fluorine, chlorine, bromine or iodine.

The term “acyloxy or acylamino containing from 1 to 10 carbon atoms” as used herein refers to a monovalent or divalent substituent comprising an alkyl group containing from 1 to 10 carbon atoms linked through a carbonyloxy or oxycarbonyl group such as a ethylcarbonyloxy, ethyloxycarbonyl group or methylcarbonyloxy, methyloxycarbonyl or linked through an aminocarbonyl or carbonylamino group such as a methylaminocarbonyl, methylcarbonylamino or ethylaminocarbonyl, ethylcarbonylamino group.

The term “alkylsulfonyl containing from 1 to 10 carbon atoms” as used herein refers to a monovalent substituent comprising a alkyl group containing from 1 to 10 carbon atoms linked through a sulfonyl group such as e.g., N-butylsulfonyl, sec-butylsulfonyl, tert-butylsulfonyl, ethylsulfonyl, n-hexylsulfonyl, isopropylsulfonyl, 2,2-dimethylpropylsulfonyl, isobutylsulfonyl, 2-methylbutylsulfonyl, 3-methyl-butylsulfonyl, 4-methylpentylsulfonyl, methyl-sulfonyl, neopentylsulfonyl, n-pentylsulfonyl, n-propylsulfonyl.

The term “one or two substituents independently selected from” shall more preferably refer to one substituent.

The term “optionally substituted hydrocarbon linkage containing from 2 to 20 carbon atoms” as used herein refers to one or more linear or branched alkyl chains, optionally substituted with, for example, alkoxy or hydroxy groups containing from 1 to 10 carbon atoms, and optionally linked together by amide, diazines, amino, ester, ether, or pyridines sulphonamide, or thioether, triazines providing a linkage.

In a preferred embodiment of the invention, M represents optionally activated agarose, silica, cellulose, glass, toyopearl, hydroxyethylmethacrylate, polyacrylamide, styrenedivinylbenzene, Hyper D, perfluorocarbons, metal oxide, glass or silica matrices, optionally coated with an organic polymer by reacting the optionally coated metal oxide, glass or silica matrix with an activating agent.

There exists a considerable number of activating agents for attaching ligands to support matrices (e.g., sepharose). The procedures by which such activating and immobilization steps carried out are well known to those skilled in the art.

In a preferred embodiment of the invention, L represents a 1,1-carbonyldiimidazole activated linkage, cyanuric bromide activated linkage, cyanuric chloride activated linkage, cyanuric floride activated linkage, cyanogen bromide activated linkage, epoxy activated linkage, 2-fluoro-1-methylpyridiniumtoluene-4-sulphonate activated linkage, glycidoxypropyltrimethoxysilane activated linkage, sodium meta-periodate activated linkage, sulphonylchloride activated linkage, tosyl activated linkage, tresyl activated linkage, 2,2,2-trifluoroethanesulphonyl chloride activated linkage, or vinylsulphone activated linkage.

In a preferred embodiment of the invention, L represents N-ethoxycarbonyl-2-ethoxy-1,2-dihydroquinoline, dicyclohexyl carbodiimide and 1-ethyl-3-(3-dimethylaminopropyl)-carbodiimide.

In one embodiment of the invention, examples of R1 and R2 (e.g., ligand compounds) include amino compounds before immobilization, such as 4-acetoxybenzylamine, 4-acetylaminobenzylamine, 1-adamantanamine, 3-aminoacetophenone, 4-aminoacetophenone, 1-aminoanthraquinone, p-aminoacetanilide, 9-aminoacridine, 4-aminoazobenzene, o-aminoazotoluene, p-aminobenzamide, 4-aminobenzamidine, p-aminobenzenesulphonamide, 2-aminobenzimidazole, o-aminobenzoic acid, m-aminobenzoic acid, p-aminobenzoic acid, 2-aminobenzothiazole, 4-aminobiphenyl, 6-amino caproic acid, 2-amino-5-chlorobenzoxazole, 4-amino-2,6-dinitrotoluene, 6-aminoindazole, 5-amino isophthalic acid, 1-amino-2-naphthol, 1-amino-3-naphthol, 1-amino-4-naphthol, 1-amino-5-naphthol, 1-amino-6-naphthol, 1-amino-7-naphthol, 1-amino-8-naphthol, 2-amino-1-naphthol, 2-amino-3-naphthol, 2-amino-4-naphthol, 2-amino-5-naphthol, 2-amino-6-naphthol, 2-amino-7-naphthol, 2-amino-8-naphthol, 1-amino-2-naphthol-4-sulfonic acid, 5-amino-1-naphthol-3-sulphonic acid, 6-amino-1-naphthol-3-sulphonic acid, 7-amino-1-naphthol-3-sulphonic acid, 1,8-diamino-3,6-dioxactane, 4-amino salicylic acid, 2-amino terephthalic acid, 2-amino-3-nitrophenol, 4-amino-2-nitrophenol, 2-aminophenol, 3-aminophenol, 4-aminophenol, 5-amino-1-phenylpyrazole, 3-aminophthalhydrazide, 3-amino-1-propanol, 2-aminopyridine, 3-aminopyridine, 4-amine-salicylic acid, amylamine, aniline, aniline-2,5-disulphonic acid, m-anisidine, o-anisidine, p-anisidine, arginine, asparagine, aspartic acid, benzidine, benzylamine, n-benzylaniline, n-benzyl-2-phenylethylamine, n-benzyltyramine, n-tert-butylaniline, p-butylaniline, n-butyl-benzylamine, p-chloroaniline, cyclohexylamine, cysteine, dehydroabietylamine, 2,4-diaminoanisole, m-diamino benzene, 1,4-diaminobutane, 4,4-diaminodiphenylmethane, 1,6-diamino hexane, 2,4-diamino-6-methylphenol, 2,4-diaminotoluene, o-dianisidine, 2,3-dichloroaniline, 2,4-dichloroaniline, 2,5-dichloroaniline, 2,6-dichloroaniline, 3,3-dichlorobenzidine, diethanolamine, diisopropylamine, 2,4-dimethylaniline, 2,6-dimethylaniline, 3,5-dinitroaniline, 2,6-diethylaniline, 2,4-dimethylaniline, 2,4-dinitroaniline, 2,6-dinitroaniline, ethanolamine, p-ethoxyaniline, ethylamine, n-ethylaniline, ethylenediamine, 2-furfurylamine, glutamic acid, glutamine, hexylamine, histamine, histidine, 4-hydroxybenzylamine, n-(2-hydroxyethyl)-benzylamine, iminodiacetic acid, isobutylamine, isoleucine, isopropylamine, 4-isopropylaniline, n-isopropylaniline, leucine, lysine, metanilic acid, methionine, 4-methoxyaniline-2-sulphoric acid, 2-methoxy-5-methylaniline, n-methylaniline, 4-methylbenzylamine, 4-methyl-3-nitroaniline, 3-methoxypropylamine, methyl 2-aminobenzoate, 4-methoxybenzylamine, 4-methylaniline-2-sulphonic acid, n-methylbenzylamine, 4,4-methylene-bis(2-chloroaniline), 4,4-methylene-bis(2-methylaniline), n-methylmetanilic acid, 2-methyl-3-nitroaniline, 2-methyl-4-nitroaniline, 2-methyl-5-nitroaniline, 2-methyl-6-nitroaniline, 3-methyl-o-phenylenediamine, 4-methyl-m-phenylenediamine, 4-methyl-o-phenylenediamine, 1-naphthylamine, 2-naphthylamine, 2-nitroaniline, 3-nitroaniline, 4-nitroaniline, octylamine, orthanilic acid, 4,4-oxydianiline 2-phenethylamine, phenylalanine, 1,4-phenylenediamine, 4-phthalazinedione, proline, propylamine, serine, sulphanilic acid, 4,4-thiodianiline, threonine, o-toluidine, p-toluidine, m-toluidine, 2,4,6-trichloroaniline, tryptamine, trptophan, tyramine, tyromine, or valine.

In another embodiment of the invention, examples of R1 and R2 include thiols such as ethylthiol, thio-p-cresol, thioglycollic acid, thiophenol. In a particular embodiment, R1 and R2 are ligands of Table 1.

In one embodiment, the invention pertains, at least in part, to a method of separation of proteins or peptides from a mixture. The method includes comprising contacting the mixture with a affinity ligand matrix library; allowing a protein- or peptide-affinity ligand complex to form; and removing the protein- or peptide-affinity ligand complex from the mixture, such that proteins or peptides are separated from said mixture.

In a further embodiment, the protein is an antibody, hormone, receptor, cell adhesion molecule, extracellular matrix molecule, immunoglobulin structure, integrin, selectin, receptor, signaling protein, growth factor, cadherin, glycoprotein, motor protein, protein phosphatase, protein kinase, enzyme, or any other protein.

In certain embodiments, the methods of the invention can be used as a method to remove or otherwise separate particular fractions in protein extracts, lysates, or other preparations or samples. The methods can be used, in certain embodiments, to remove proteins or other macromolecules of lesser interest from biologically active proteins or other proteins of interest from a particular mixture. In this way, the methods of the inventions can, in certain embodiments, be used to fractionate proteins, such is currently done through methods such as chromatography or sucrose gradients.

In a further embodiment, the initial mixture is plasma, blood, protein extract, protein lysate, a biological sample, or any proteinacious sample.

The term “affinity ligand matrix library” includes libraries of small molecule ligands optionally bound to a solid phase matrix, as described above. The affinity ligand matrix library also may comprise active groups bound to a label. Preferably, the labels allow the protein-affinity ligand matrix complexes to be selectively removed from the mixture.

In another embodiment, the methods of the invention also pertain to a method of depleting one or more peptides or proteins from a mixture. The method includes contacting a mixture with a chemically reactive matrix to form a protein or peptide complex with said chemically reactive matrix, removing the protein or peptide complexes, such that said proteins or peptides are depleted from the mixture.

In another embodiment, the invention pertains to a method of identifying a selectively reactive affinity ligand for a protein. The method includes contacting the protein with an affinity ligand matrix library, allowing one or more protein complexes to form with said selectively reactive affinity ligand, and identifying the protein complex, such that the selectively reactive affinity ligand for the protein is identified.

The term “selectively reactive affinity ligand” refers to a ligand that preferentially binds to a protein or peptide of interest. Preferably, the ligand binds to the protein or peptide at a rate 2 times, 3 times, 4 times, 10 times, or 20 times that of binding to other proteins in the mixture. In certain embodiments, the peptide binds to a selectively reactive affinity ligand at a rate which allows it to be separated from the majority of the molecules in the mixture.

Once the selectively reactive affinity ligand for a particular protein or peptide is identified it can be used to separate out that protein from other mixtures. In this way, the methods of the invention can be used to obtain specific proteins of interest from a complex mixture using particular reactive affinity ligands. This is of particular interest for therapeutic proteins or other proteins of particular interest which are obtained from biological samples or other complex mixtures.

In another embodiment, the invention also pertains, at least in part, to a method of selectively obtaining a protein of interest from a mixture. The method includes contacting a mixture with a selectively reactive affinity ligand such that a protein complex with the protein of interest and the selectively reactive affinity ligand is formed, and separating the protein complex from the mixture. The selectively reactive affinity ligands can be identified using the methods described above.

### Therapeutic Use of Low-Abundance Proteins Identified by the Present Invention

Proteins have been evolutionarily selected to perform specific functions. Thus, the identification of low-abundance proteins has tremendous potential as a biological tool for studying cellular processes as well as for developing novel and specific therapeutic agents. Many natural proteins are currently being used to treat diabetes, anemia, hepatitis and cancer.

The present invention provides a method for the isolation of low-abundance proteins or the depletion of high-abundance proteins. Accordingly, in one embodiment, the present invention also provides a method for the isolation of biopharmaceutical or medicinal proteins and/or peptides from a proteinacious sample of interest. In a further embodiment, the present invention provides a method from the identification of a change in protein expression, e.g., in a diseased state a protein that was a high-abundance protein in a normal sample may become a low-abundance protein, or a low-abundance protein in a normal sample may become a high-abundance protein in a diseased sample. In another embodiment, proteins isolated by the methods of the present invention can be used as therapeutic agents to treat a subject with a disease. In a further embodiment, the isolation of proteins by the methods of the present invention can be used as a diagnostic tool for identifying a subject with a disease or a diseased sample. In yet another embodiment, the proteins isolated by the methods of the present invention can be used as diagnostic agents to identify a subject with a disease or a diseased sample.

In one embodiment, the disease may be cancer, an autoimmune disorder, a neurological disorder, an infectious disease, heart disease, a genetic disease, a behavioral disorder, or a mental disorder. In another embodiment, the cancer may be a carcinoma or a sarcoma. In yet another embodiment the cancer may be breast cancer, brain cancer, prostate cancer, bladder cancer, liver cancer, lymphoma, pancreatic cancer, stomach cancer, colon cancer, or skin cancer (e.g., melanoma).

The invention will now be described in further detail with reference to the following examples. The examples are provided for illustrative purposes, and are not to be construed as limiting the scope of the invention in any way.

### Exemplary Embodiments

General methods for preparing the general affinity ligand matrix library of the invention can be found in Dong et al., Utilizing a library of synthetic affinity ligands for the enrichment, depletion and one-step purification of leech proteins. J. Mol. Recognit. 2008; 21: 163-168; which is incorporated herein by reference in its entirety.

### Example 1

### Synthesis of a Typical Affinity Matrix (Epichlorohydrin as the Scaffold)

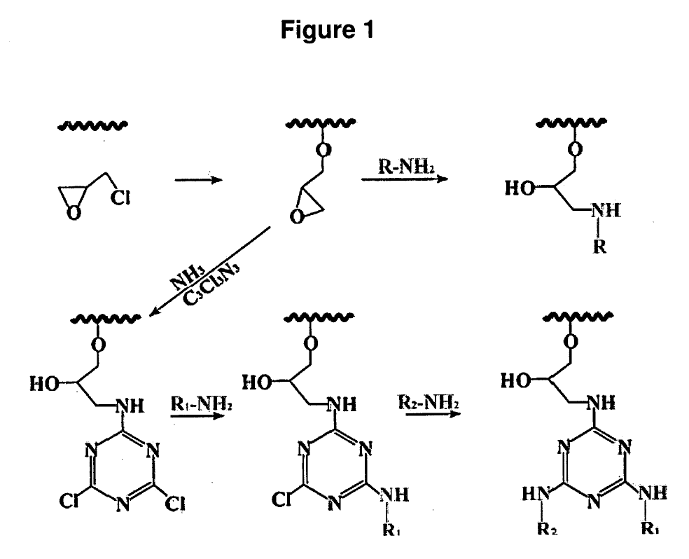

Sepharose 4B (100 g) was thoroughly washed with water at a 10:1 ratio, drained, and suspended in 50 ml activating solution (1 M NaOH, 2.5 g sodium borohydride, and 10 ml epichlorohydrin). The mixture was tumbled for 2 h at 60° C. The activated gel was then washed thoroughly with 10:1 distilled water until the Ph of the eluate was 7.0. After draining, 20.0 g activated Sepharose 4B was placed into a glass bottle with a stopper. 0.5 g amino compounds (e.g., 2-aminobenzimidazole) (Table 1) was dissolved with 25 ml 1,4-dioxane contained 0.1M NaOH, then transferred into the same bottle. The mixture tumbled for 24 h at 60° C. Mercaptoethanol (1.0 ml) was then added into the bottle and continued to tumble for 2 h at 60° C. Finally, the gel was washed thoroughly with 10:1 distilled water until the Ph of eluate solution was 7.0 and suspended in 20% (v/v) ethanol for storage (FIG. 1). The density of the epoxy groups on the activated agarose was measured with the following procedure: 1.0 g epoxy derivative was thoroughly washed, drained, and suspended in 3 ml of 1.3 M sodium thiosulphate solution, and the mixture was incubated for 20 min at room temperature, and then titrated to ph 7.0 by the addition of 0.1 M HCl. The epoxy group density in μmol/g gel is equal to the number of moles of acid required for the titration.

TABLE 1
Amines used for the construction of the ligand library

| No. | Amines |
|---|---|
| 1 | 3-amino-1-propanol |
| 2 | 3-methoxypropylamine |
| 3 | ethanolamine |
| 4 | ethylenediamine |
| 5 | 4-methyl-3-nitroaniline |
| 6 | octylamine |
| 7 | propylamine |
| 8 | 1,4-phenylenediamine |
| 9 | o-toluidine |
| 10 | p-toluidine |
| 11 | 2,4,6-trichloroaniline |
| 12 | m-toluidine |
| 13 | 2,3-dichloroaniline |
| 14 | methyl 2-aminobenzoate |
| 15 | m-anisidine |
| 16 | o-anisidine |
| 17 | 2,4-diamino-6-methylphenol |
| 18 | 1-naphthylamine |
| 19 | 2,4-diaminotoluene |
| 20 | 3′-aminoacetophenone |
| 21 | 2,4-dimethylaniline |
| 22 | 2,4-dinitroaniline |
| 23 | 4′-aminoacetophenone |
| 24 | 2,6-dichloroaniline |
| 25 | 2,6-diethylaniline |
| 26 | 4-amino-2-nitrophenol |
| 27 | 2,6-dinitroaniline |
| 28 | 2-aminophenol |
| 29 | 2-amino-3-nitrophenol |
| 30 | 2-aminopyridine |
| 31 | 2-naphthylamine |
| 32 | 2-methoxy-5-methylaniline |
| 33 | 2-nitroaniline |
| 34 | 3-aminopyridine |
| 35 | 2-methyl-3-nitroaniline |
| 36 | 3-aminophenol |
| 37 | 4-aminophenol |
| 38 | 2-methyl-4-nitroaniline |
| 39 | 3-nitroaniline |
| 40 | 4-nitroaniline |
| 41 | 2-methyl-6-nitroaniline |
| 42 | 2-phenethylamine |
| 43 | 3,5-dinitroaniline |
| 44 | 2-methyl-5-nitroaniline |
| 45 | 2,4-dichloroaniline |
| 46 | 2-aminobenzothiazole |
| 47 | 3-methyl-o-phenylenediamine |
| 48 | 2,5-dichloroaniline |
| 49 | 4,4′-thiodianiline |
| 50 | 4,4′-diaminodiphenylmethane |
| 51 | 4,4′-oxydianiline |
| 52 | 2,6-dimethylaniline |
| 53 | 4,4′-methylene-bis(2-chloroaniline) |
| 54 | 4-aminobiphenyl |
| 55 | 4-aminobenzoic acid |
| 56 | 4,4′-methylene-bis(2-methylaniline) |
| 57 | 4-aminoazobenzene |
| 58 | aniline |
| 59 | 4-amino-2,6-dinitrotoluene |
| 60 | isopropylamine |
| 61 | benzidine |
| 62 | 4-methyl-m-phenylenediamine |
| 63 | 4-isopropylaniline |
| 64 | benzylamine |
| 65 | 4-methyl-o-phenylenediamine |
| 66 | p-anisidine |
| 67 | o-aminoazotoluene |
| 68 | 1-amino-2-naphthol-4-sulfonic acid |
| 69 | 2,4-diaminoanisole |
| 70 | o-dianisidine |
| 71 | 2-aminobenzimidazole |
| 72 | 6-amino caproic acid |
| 73 | 2-furfurylamine |
| 74 | 5-aminoisophthalic acid |
| 75 | 4-phthalazinedione |
| 76 | cyclohexylamine |
| 77 | 1-aminoanthraquinone |
| 78 | 4-aminosalicylic acid |
| 79 | arginine |
| 80 | 2-aminoterephthalic acid |
| 81 | 9-aminoacridine |
| 82 | tryptamine |
| 83 | 3,3′-dichlorobenzidine |

### Example 2

### Synthesis of Another Typical Affinity Matrix (Cyanuric Chloride as the Scaffold)

After 100 g Sepharose 4B was activated by epichlorohydrin according to the procedure mentioned above, the activated Sepharose 4B was suspended in 350 ml distilled water, and 150 ml 35% ammonia was added. The gel was incubated for 12 h at 30° C. on a rotary shaker (200 rpm), after which it was thoroughly washed with distilled water on a sinter funnel. Aminated Sepharose 4B was suspended in 350 ml 50% (v/v) acetone solution, maintained at 0° C. in an ice-salt bath and 8 g cyanuric chloride (44 mmol) dissolved in 70 ml acetone was added over a period of 2 h with shaking; Ph was maintained at neutral by the addition of 1 M NaOH. A ninhydrin test was performed to indicate the existence of free amino groups on aminated Sepharose 4B and the linkage of cyanuric chloride in the following procedure: a small aliquot of gel was smeared on filter paper, sprayed with ninhydrin solution [0.2% (w/v) in acetone], and heated briefly with a hair dryer. Purple color indicated the presence of free amino groups and color disappearance indicated that cyanuric chloride had been linked to the amino groups. Ninhydrin colorimetry can be used to measure the density of free amino groups on aminated Sepharose 4B and the linkage of cyanuric chloride to the amino groups. The gel was sequentially washed with 50% (v/v) acetone. A portion of the 20 g dichlorotriazinyl wet gel was reacted with a 1.1-fold molar excess of an aminated compound (e.g., 2-aminobenzothiazole) at 50° C. (R1 substitution). After washing, a portion of the R1 substituted wet gel was reacted with a 5-fold molar excess of an aminated compound (e.g., 4-aminobiphenyl 4-aminobenzoic acid) at 95° C. (R2 substitution) (FIG. 1). NaHCO₃ (5% w/v) was used to maintain the ph between 6.0 and 7.0 during the reaction. Finally, the gel was washed with dimethylformamide then distilled water, drained, and suspended in 20% (v/v) ethanol for storage.

Seven hundred columns of synthetic ligands were constructed.

### Nomenclature Regarding Column Identification

If only one compound (ligand) was immobilized to the base matrix (M) through a scaffold, the column was named “A,” followed by the compound number. If two compounds (ligands) were immobilized to the base matrix (M) through a scaffold the column was named “A” followed by the two compound numbers connected with a dash (“-”). Although all of the compounds of Table 1 were used to create the general affinity ligand matrix library, the column numbers used for identification do not necessarily correspond with the numbers used to list the compounds in Table 1.

### Example 3

### Use of the Synthetic Affinity Matrix Library to Treat Leech Protein (Screening of Solid Phase Supported Ligands for Protein Binding)

Of the seven hundred columns of synthetic ligands that were constructed, two hundred and ninety-seven columns were selected to bind leech protein. The two hundred ninety-seven affinity matrix Gels (1 ml) were packed into small polystyrene columns with porous discs at both the bottom and the top of the gel to bind leech extract proteins. The columns were equilibrated with a 10-fold column volume of 10 mm phosphate buffer (Ph 7.0). The crude extract of leech was loaded onto the columns. Phosphate buffer (10 mm) was applied to elute out the flow-through fraction until the baseline of UV monitor went down to the bottom. The flow-through eluate was collected. After that, 0.1 M acetic acid was applied to elute out the fraction of bound protein. Finally, 0.1 M NaOH plus 30% ethanol was applied to wash and clean the column until the baseline of UV monitor went down to the bottom. All the eluates collected were used for subsequent SDS-PAGE electrophoresis.

After SDS-PAGE analysis of the collected fractions, it was found that: (1) twenty-five columns presented high binding and recovery of leech proteins. They could bind more than 70% of the leech protein; (2) fifty-eight columns could bind all of the proteins except for one or several missing abundant proteins. For instance, the band profile of column A8-21 was almost the same as the crude extract except a band of a 19.1 kd abundant protein was missing; (3) Twenty-four columns could absorb less than 1% of leech protein; (4) One hundred fifty-four columns could enrich some specific proteins. For instance, 6 columns could enrich the 102.2 kd protein, 8 columns could enrich the 32.5 kd protein, 15 columns could enrich the 30.0 kd protein, 7 columns could enrich the 25.4 kd protein, 10 columns could enrich the 32.0-43.7 kd protein bands, 13 column could enrich the less than 14.2 kd protein bands, and 67 columns could enrich several protein bands that could clearly be distinguished; (5) Thirty-six columns showed an apparent one-step purification effect on the crude extract of leech.

### Example 4

### Use of the Synthetic Affinity Matrix Library to Enrich Low-Abundance Proteins from Leech Extract

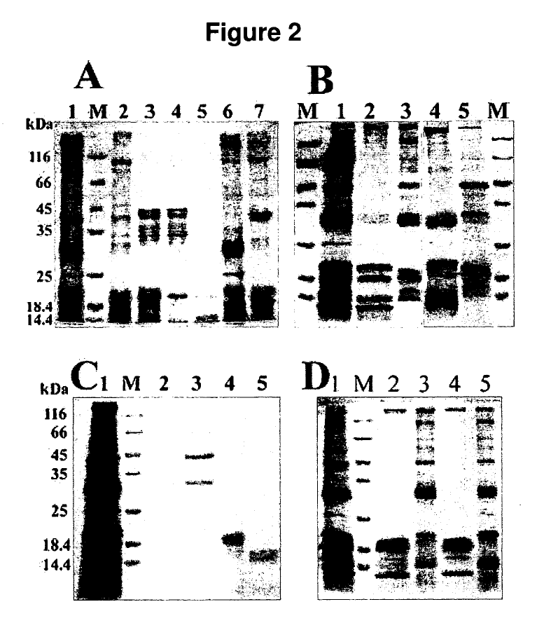

The eluates of several typical columns of the 154 columns that enrich specific proteins are shown run on a 12% SDS-PAGE gel in FIG. 2A. Crude extracts were concentrated five-fold and the column eluates were concentrated 10-fold. Lane 1 shows the crude extract of leech.

### Column A8-47

Low-abundance protein (1.0%) of 102.2 and 87.0 kd could be four-fold concentrated by column A8-47. Low-abundance protein (0.1-0.2%) of 43.7 and 34.1 kd could be concentrated to 2.3% abundance (FIG. 2A, Lane 2). The effective structure of A8-47 was aniline and 1,8-diamino-3,6-dioxactane immobilized through cyanuric chloride activation chemistry.

### Columns A29-32 and A6-2

A29-32 (FIG. 2A, Lane 3) and A6-2 (FIG. 2A, Lane 4) had very similar profiles and primarily enriched for the low-abundance proteins ranging from 32.0 to 43.7 kd. The proteins could be enriched 20- to 40-fold and reach 4.0% abundance. An apparent difference between these two columns was that the binding of column A29-32 to the 19.1 kd band was greater than that of A6-2. The structure of A6-2 was 1,6-diamino hexane and hexylamine immobilized through cyanuric chloride activation chemistry. The structure of A29-32 was m-amino phenol and m-diamino benzene immobilized through cyanuric chloride activation chemistry.

### Column A4-11

Column A4-11 (FIG. 2A, Lane 5), can enrich the band at 32.5 kd. The protein concentration could be elevated from 1.7 to 25.0%, and concentrated 15-fold.

### Column A11-70

Column A11-70 (FIG. 2A, Lane 6), enriched the 30.0 kd band from 10.5 to 24.0%, the 25.4 kd band from 0.86 to 2.6%, the 21.8 kd band from 1.0 to 6.2%, and the 15.2 kd band from 18.1 to 50.0%. The structure of A11-70 was 4-Aminobenzamidine and L-Proline immobilized through cyanuric chloride activation chemistry.

### Column A2

Column A2 (FIG. 2A, Lane 7), enriched the 41.0 kD band from 3.85 to 9.5%.

### Example 5

### Use of the Synthetic Affinity Matrix Library to Deplete High Abundance Proteins of Leech Extract

Approximately 83 of the columns can be used for the depletion of high abundance proteins. Twenty-five of these columns were found to bind more than 70% of the leech protein. The band profile of bound proteins was similar to the crude extract of leech.

### Column A6

Column A6 was used for depleting abundant proteins (FIG. 2B, Lanes 4 and 5). Lane 1 is crude extract and lane 2 shows the flow through (FIG. 2B). About 50% of the total protein was removed from the crude extract by one-step depletion using ligand A6. Abundant proteins of 19.1, 31.0, and 43.0 kd molecular masses were depleted the most. The band profile of the flow through fraction showed low abundance proteins. A6 is 1,6-diamino hexane immobilized with epoxide.

### Example 6

### Use of the Synthetic Affinity Matrix Library for One-Step Purification One-Step Purification

Thirty-six columns showed an apparent one-step purification effect on the crude extract of leech. Lane 1 of FIG. 2C shows the crude extract and Lane 2 shows the eluate of column A903. A band of 32.5 kD could be purified in one step from 0.6 to 90%. Lane 3 shows that column NX could simultaneously purify bands of 65.2, 44.0, and 31.5 kD molecular mass, and raise the protein contents from 0.6%, 3.0%, and 5.0% to 4.6%, 45.0%, and 35.0%, respectively. Lane 4 shows that columns A83-83 could purify the abundant protein of 19.1 kD molecular mass and raise the protein content from 28.0% to 72.0%. Using the acid gradient elution, the 19.1 kD protein could be purified to an electrophoretically pure grade. Lane 5 shows the column A32-53 could purify the 14.9 kD mass protein and raise its content from 3.9 to 75.0%. The reason for band diffusion might be strong ionic effect of the electrophoretic buffer or that the polymer was a small peptide.

### One-Step Purification Via Flow-Through

Furthermore, the flow-through from the 58 columns that bound all but one or more of the abundant proteins could be used to purify these proteins in one step. The band profiles of the flow-through demonstrated the existence of the missing band (FIG. 2D).

### Example 7

### Synthetic Affinity Matrix Library

Sepharose 4B (500 g) was washed with 10× gel volume of dH₂O, mixed with 50 mL epichlorohydrin, 2.5 g Sodium Borohydride and NaOH(1.0 M, 200 mL), and then dried. The reaction was continued for 2 h at 60° C. Then the gel was washed with dH₂O to neutral, and the epoxide density was measured by titration of the OH⁻ librated when reacted with sodium thiosulfate, is 25 μmol/g wet gel. 2-aminoterephthalic acid (1.0 g) was dissolved in 0.1 M NaOH, and mixed with a portion of 10 g activated wet gel. The mixture was kept at 60° C. overnight, and the media washed with 10× gel volume of dH₂O, 3 volume of 30% ethanol for storage. The density of immobilized 2-aminoterephthalic acid was measured by dissolving 0.5 g gel in HCl (1.0M) at 95° C., and measuring the absorbance at 20 μmol/ml.

### Example 8

### Use of the Synthetic Affinity Matrix Library to Process Human Plasma

Human plasma (100 mL), centrifuged at 3000×g for 10 min at 4° C. to remove cell debris, was conditioned to phosphate buffer (20 mm, ph 7.0). 4 mL were loaded onto a library of 450 affinity columns (1 mL), equilibrated with 10 ml phophate buffer (20 mm, ph 7.0), and washed with 10 ml phophate buffer (20 mm, ph 7.0) to remove unabsorbed proteins, and then eluted with 5 ml acetic acid (0.1 M), finally cleaned with 0.1 M NaOH. All fractions were collected and analyzed with Reducing 12% SDS-PAGE and revealed by Coomassie-blue staining.

### Example 9

### Use of the Synthetic Affinity Matrix Library to Enrich Low-Abundance Proteins of Human Plasma

### Column 8-1

Column 8-1 could bind low abundance proteins and enriched more than 25 low abundance proteins clearly visible by SDS-PAGE analysis. The enrichment process also removed the masking effect of high abundance proteins. Column 8-1 was aniline and butylamine immobilized through cyanuric chloride activation chemistry.

### Example 10

### Use of the Synthetic Affinity Matrix Library to Deplete High Abundance Proteins Of Human Plasma

### Column 10-2

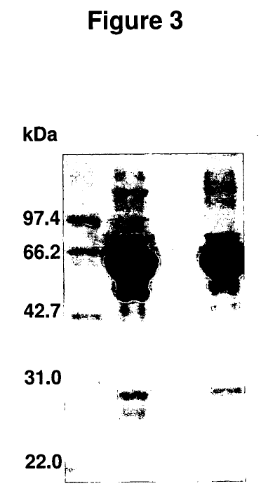

Column 10-2 could bind all the high abundance proteins in human plasma, including protein bands with molecular weight of about 139, 84, 66, 60, 50, 47, 25, 16 kd, these band included albumin, and antibody heavy and light chains. The flow-through fractions included the low abundance proteins of interest (FIG. 3). Column 10-2 was 3-aminophthalhydrazide and L-phenylalanine immobilized through cyanuric chloride activation chemistry.

### Example 11

### Use of the Synthetic Affinity Matrix Library to Enrich Low Abundance Proteins of Human Plasma

### Column 12-6

Column 12-6 bound low abundance proteins and enriched more than 20 low abundance proteins visible by SDS-PAGE analysis. The enrichment process also removed the masking effect of high-abundance proteins. Column 12-6 was dehydroabietylamine and 1,6-diamino hexane immobilized through cyanuric chloride activation chemistry.

### Column 12-9

Column 12-9 bound low abundance proteins and enriched more than 10 low abundance proteins visible by SDS-PAGE analysis. The enrichment process also removed the masking effect of high abundance proteins. Column 12-9 was dehydroabietylamine and cyclohexylamine immobilized through cyanuric chloride activation chemistry.

### Column 13-3

Column 13-3 bound low abundance proteins, and enriched about 15 low abundance proteins visible by SDS-PAGE gel analysis. The enrichment process also removed the masking effect of high abundance proteins. Column 13-3 was 3-picolylamine and Octylamine immobilized through cyanuric chloride activation chemistry.

### Column 13-9

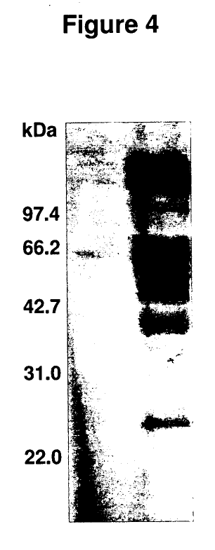

Column 13-9 bound more than 15 low abundance proteins visible by SDS-PAGE analysis. The enrichment process also removed the masking effect of other high abundance proteins. The enriching effect of low abundance proteins is very apparent (FIG. 4). Column 13-9 was 3-picolylamine and cyclohexylamine immobilized through cyanuric chloride activation chemistry.

### Column 13-1

Column 13-1 bound more than 25 low abundance proteins visible by SDS-PAGE analysis. The enrichment process also removed the masking effects of other high abundance proteins. The enriching effect of low abundance proteins is very apparent. Column 13-11 was 3-picolylamine and 4-aminobenzamidine immobilized through cyanuric chloride activation chemistry.

### Column 13-18

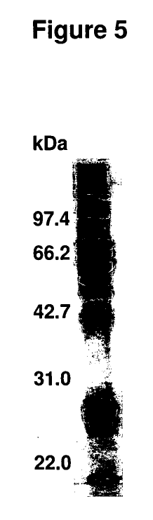

Column 13-18 bound low abundance proteins and enriched about 20 low abundance proteins visible by SDS-PAGE analysis. The enrichment process also removed the masking effect of high abundance proteins (FIG. 5). Column 13-18 was 3 picolylamine and 2-aminoterephthalic acid immobilized through cyanuric chloride activation chemistry.

### Column 14-11

Column 14-11 bound low abundance proteins and enriched about 20 low abundance proteins visible by SDS-PAGE analysis. The enrichment process also removed the masking effect of high abundance proteins. Column 14-11 was 1-adamantanamine and 4-aminobenzamidine immobilized through cyanuric chloride activation chemistry.

### Column 16-8

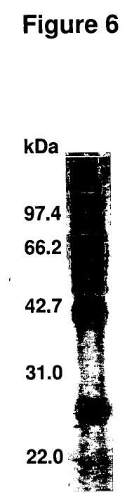

Column 16-8 bound low abundance proteins in the existence of more than 7 high abundance proteins such as albumin and antibody, and enriched low abundance proteins visible without the masking effects of high abundance proteins (FIG. 6). Column 16-8 was 4-amine-salicylic acid and aniline immobilized through cyanuric chloride activation chemistry.

### Column 23-11

Column 23-11 bound about 7 low abundance proteins in the existence of high abundance proteins, and enriched low abundance proteins, visible by SDS-PAGE analysis. Column 23-11 was iminodiacetic acid and 4-aminobenzamidine immobilized through cyanuric chloride activation chemistry.

### Column 55-1

Column 55-1 bound low abundance proteins and enriched more than 17 low abundance proteins visible by SDS-PAGE analysis. The enrichment process also removed the masking effect of high abundance proteins. Column 55-1 was lysine and butylamine immobilized through cyanuric chloride activation chemistry.

### Column 55-8

Column 55-8 bound low abundance proteins and enriched more than 22 low abundance proteins visible by SDS-PAGE analysis. The enrichment process also removed the masking effect of high abundance proteins. Column 55-8 was lysine and aniline immobilized through cyanuric chloride activation chemistry.

### Column 55-22

Column 55-22 bound low abundance proteins and enriched more than 16 low abundance proteins visible by SDS-PAGE analysis. The enrichment process also removed the masking effect of high abundance proteins. Column 55-22 was lysine and 1-aminoanthroquinone immobilized through cyanuric chloride activation chemistry.

### Example 12

### Use of the Synthetic Affinity Matrix Library to Deplete High Abundance Proteins Of Human Plasma

### Column 17-17

Column 17-17 bound all the high abundance proteins in human plasma, leaving the low abundance proteins to be further studied. Column 17-17 comprised two moles of 5-Amine-Isophthalic Acid immobilized through cyanuric chloride activation chemistry.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

1. A general affinity ligand matrix library comprising at least one ligand compound with an active group selected from the group consisting of (a) an amino group, (b) a sulfhydryl group, (c) a hydroxyl group, (d) a carbonyl group, and (e) an active hydrogen group.

2. The general affinity ligand matrix library of claim 1, wherein said library comprises approximately 300-1000 ligands.

3. The general affinity ligand matrix library of claim 1, wherein said library comprises approximately 700 ligands.

4. The general affinity ligand matrix library of claim 1, wherein said library comprises synthetic ligands.

5. The general affinity ligand matrix library of claim 1, wherein said library comprises a base matrix.

6. The general affinity ligand matrix library according to claim 5, wherein the base matrix is any compound or material, and wherein the material is particulate or non particulate, soluble or insoluble, or porous or nonporous.

7. The general affinity ligand matrix library according to claim 5, wherein the base matrix comprises activated agarose, silica, cellulose, glass, toyopearl, hydroxyethylmethacrylate, polyacrylamide, styrenedivinylbenzene, Hyper D, or perfluorocarbons.

8. The general affinity ligand matrix library according to claim 1 wherein at least one ligand compound comprises a saturated or unsaturated, linear, branched, or ring alkyl compound containing from 1 to 10 carbon or hetero atoms.

9. (canceled)

10. The general affinity ligand matrix library according to claim 1, wherein at least one ligand compound is linear or ring, saturated or unsaturated, containing from 2 to 20 carbon atoms, 0-6 nitrogen atoms, 0-6 oxygen atoms, 0-3 sulfur atoms, or 0-3 phosphorous atoms.

11. The general affinity ligand matrix library according to claim 1, wherein at least one ligand compound is an alkyl.

12. (canceled)

13. (canceled)

14. The general affinity ligand matrix library according to claim 10, wherein at least one of the ligand compound is benzimidazole, benzoxazole, benzthiazole, catechol, p-chlorophenol o-cresol, m-cresol, p-cresol, hydroquinone, 2-hydroxy-3-naphthoic acid, indazole, naphthalene, 1-naphthol, 2-naphthol, 1-naphthol-4-sulphonic acid, 2-naphthol-6-sulphonic acid, resorcinol, indazole, naphthyl, phenyl, phenol, or 1-phenylpyrazole ring, and wherein these rings are optionally substituted with one or more substituents.

15. (canceled)

16. The general affinity ligand matrix library according to claim 1, wherein at least one of the ligand compounds comprises an amino group.

17. (canceled)

18. The general affinity ligand matrix library according to claim 5, wherein at least one of said ligand compounds is attached to said base matrix through activation and immobilization through amine reactive chemistry, sulfhydryl reactive chemistry, carbonyl chemistry, hydroxyl reactive chemistry, active hydrogen reactive chemistry, or photoreactive reactive chemistry.

19. The general affinity ligand matrix library of claim 18, wherein the sulfhydryl reactive chemistry utilizes cyanogen bromide, carbonyl diimidazole, divinylsulfone, azlactone, cyanuric chloride, 2-fluoro-1-methylpyridinium toluene-4-sulfonate, tosyl chloride, tresyl chloride, iodoacetyl, bromoacetyl, maleimide, pyridyl disulfide, epoxyl, bisoxirane, or 5-thio-2-nitro benzoic acid as a reactant.

20. The general affinity ligand matrix library of claim 18, wherein the carbonyl chemistry utilizes hydrazide or reductive animation.

21. The general affinity ligand matrix library of claim 18, wherein the hydroxyl reactive chemistry utilizes cyanogen bromide, cyanuric chloride, epoxyl, or bisoxirane.

22. The general affinity ligand matrix library of claim 18, wherein the active hydrogen reactive chemistry utilizes a Diazonium or a Mannich Condensation.

23. The general affinity ligand matrix library claim 18, wherein the photoreactive reactive chemistry utilizes p-azidophenyl glyoxal, azidobenzoyl hydrazide, sulfosuccunimidyl-6-(4′-azido-2′-nitrophenylamino)hexanoate, or N-[4-(p-azidosalicylamido)butyl]-3′-(2′-pyridyldithio) propionamide.

24-48. (canceled)

49. A method for separating proteins or peptides from a mixture, comprising:
contacting said mixture with a general affinity ligand matrix library;
such that a protein- or peptide-affinity ligand complex forms; and
removing said protein- or peptide-affinity ligand complex from said mixture, such that proteins or peptides are separated from said mixture.

50. The method of claim 49, wherein said protein is an antibody, hormone, receptor, or a cell adhesion molecule.

51. The method of claim 49, wherein said mixture is selected from the group consisting of plasma, blood, protein extract, protein lysate, biological sample, ascites, serum, chyle, semen, interstitial fluid, lymph fluid, menses, breast milk, sweat, tears, urine, vaginal lubrication, Cowper's fluid or pre-ejaculatory fluid, female ejaculate, mucus, pleural fluid, pus, saliva, sebum (skin oil), chyme, vomit, or any proteinacious sample.

52. The method of any one of claim 49, wherein said general affinity ligand matrix library comprises a label.

53. The method of claim 49, wherein said general affinity ligand matrix library comprises a ligand with an active group.

54. The method of claim 53, wherein said active group is an amino group, a sulfhydryl group, hydroxyl group, a carbonyl group, or an active hydrogen group.

55. (canceled)

56. (canceled)

57. A method of identifying a selectively reactive affinity ligand for a protein, comprising contacting said protein with a general affinity ligand matrix library of claim 1, allowing one or more protein complexes to form with said selectively reactive affinity ligand, and identifying said protein complex, such that said selectively reactive affinity ligand for said protein is identified.

58. The method of claim 57, wherein said protein is a protein of therapeutic use.

59. (canceled)

60. (canceled)

61. A general affinity ligand matrix library, wherein the affinity matrix of each component of the library comprises the general formula: M-L-R1(R2).

62. The affinity matrix of claim 61, wherein M represents activated agarose, silica, cellulose, glass, toyopearl, hydroxyethylmethacrylate, polyacrylamide, styrenedivinylbenzene, Hyper D, perfluorocarbons, metal oxide, glass or silica matrices, optionally coated with an organic polymer.

63. The affinity matrix of claim 61, wherein L represents a 1,1′-carbonyldiimidazole activated linkage, cyanuric bromide activated linkage, cyanuric chloride activated linkage, cyanuric floride activated linkage, cyanogen bromide activated linkage, epoxy activated linkage, 2-fluoro-1-methylpyridiniumtoluene-4-sulphonate activated linkage, glycidoxypropyltrimethoxysilane activated linkage, sodium meta-periodate activated linkage, sulphonylchloride activated linkage, tosyl activated linkage, tresyl activated linkage, 2,2,2-trifluoroethanesulphonyl chloride activated linkage, or vinylsulphone activated linkage.

64. The affinity matrix of claim 61, wherein L represents N-ethoxycarbonyl-2-ethoxy-1,2-dihydroquinoline, dicyclohexyl carbodiimide and 1-ethyl-3-(3-dimethylaminopropyl)-carbodiimide.

65. The affinity matrix of claim 61, wherein R1(R2) represents the immobilization of one or two chemical groups, wherein the chemical groups are alkyl or aryl groups.

66. The affinity matrix of claim 65, wherein R1 and R2 are selected from the group comprising, 4-acetoxybenzylamine, 4-acetylaminobenzylamine, 1-adamantanamine, 3-aminoacetophenone, 4-aminoacetophenone, 1-aminoanthraquinone, p-aminoacetanilide, 9-aminoacridine, 4-aminoazobenzene, o-aminoazotoluene, p-aminobenzamide, 4-aminobenzamidine, p-aminobenzenesulphonamide, 2-aminobenzimidazole, o-aminobenzoic acid, m-aminobenzoic acid, p-aminobenzoic acid, 2-aminobenzothiazole, 4-aminobiphenyl, 6-amino caproic acid, 2-amino-5-chlorobenzoxazole, 4-amino-2,6-dinitrotoluene, 6-aminoindazole, 5-amino isophthalic acid, 1-amino-2-naphthol, 1-amino-3-naphthol, 1-amino-4-naphthol, 1-amino-5-naphthol, 1-amino-6-naphthol, 1-amino-7-naphthol, 1-amino-8-naphthol, 2-amino-1-naphthol, 2-amino-3-naphthol, 2-amino-4-naphthol, 2-amino-5-naphthol, 2-amino-6-naphthol, 2-amino-7-naphthol, 2-amino-8-naphthol, 1-amino-2-naphthol-4-sulfonic acid, 5-amino-1-naphthol-3-sulphonic acid, 6-amino-1-naphthol-3-sulphonic acid, 7-amino-1-naphthol-3-sulphonic acid, 1,8-diamino-3,6-dioxactane, 4-amino salicylic acid, 2-amino terephthalic acid, 2-amino-3-nitrophenol, 4-amino-2-nitrophenol, 2-aminophenol, 3-aminophenol, 4-aminophenol, 5-amino-1-phenylpyrazole, 3-aminophthalhydrazide, 3-amino-1-propanol, 2-aminopyridine, 3-aminopyridine, 4-amine-salicylic acid, amylamine, aniline, aniline-2,5-disulphonic acid, m-anisidine, o-anisidine, p-anisidine, arginine, asparagine, aspartic acid, benzidine, benzylamine, n-benzylaniline, n-benzyl-2-phenylethylamine, n-benzyltyramine, n-tert-butylaniline, p-butylaniline, n-butyl-benzylamine, p-chloroaniline, cyclohexylamine, cysteine, dehydroabietylamine, 2,4-diaminoanisole, m-diamino benzene, 1,4-diaminobutane, 4,4-diaminodiphenylmethane, 1,6-diamino hexane, 2,4-diamino-6-methylphenol, 2,4-diaminotoluene, o-dianisidine, 2,3-dichloroaniline, 2,4-dichloroaniline, 2,5-dichloroaniline, 2,6-dichloroaniline, 3,3-dichlorobenzidine, diethanolamine, diisopropylamine, 2,4-dimethylaniline, 2,6-dimethylaniline, 3,5-dinitroaniline, 2,6-diethylaniline, 2,4-dimethylaniline, 2,4-dinitroaniline, 2,6-dinitroaniline, ethanolamine, p-ethoxyaniline, ethylamine, n-ethylaniline, ethylenediamine, 2-furfurylamine, glutamic acid, glutamine, hexylamine, histamine, histidine, 4-hydroxybenzylamine, n-(2-hydroxyethyl)-benzylamine, iminodiacetic acid, isobutylamine, isoleucine, isopropylamine, 4-isopropylaniline, n-isopropylaniline, leucine, lysine, metanilic acid, methionine, 4-methoxyaniline-2-sulphoric acid, 2-methoxy-5-methylaniline, n-methylaniline, 4-methylbenzylamine, 4-methyl-3-nitroaniline, 3-methoxypropylamine, methyl 2-aminobenzoate, 4-methoxybenzylamine, 4-methylaniline-2-sulphonic acid, n-methylbenzylamine, 4,4′-methylene-bis(2-chloroaniline), 4,4′-methylene-bis(2-methylaniline), n-methylmetanilic acid, 2-methyl-3-nitroaniline, 2-methyl-4-nitroaniline, 2-methyl-5-nitroaniline, 2-methyl-6-nitroaniline, 3-methyl-o-phenylenediamine, 4-methyl-m-phenylenediamine, 4-methyl-o-phenylenediamine, 1-naphthylamine, 2-naphthylamine, 2-nitroaniline, 3-nitroaniline, 4-nitroaniline, octylamine, orthanilic acid, 4,4′-oxydianiline 2-phenethylamine, phenylalanine, 1,4-phenylenediamine, 4-phthalazinedione, proline, propylamine, serine, sulphanilic acid, 4,4′-thiodianiline, threonine, o-toluidine, p-toluidine, m-toluidine, 2,4,6-trichloroaniline, tryptamine, trptophan, tyramine, tyromine, and valine.

67. The affinity matrix of claim 65, wherein R1 and R2 represent thiols.

68. The affinity matrix of claim 67, wherein R1 and R2 are selected from the group comprising, ethylthiol, thio-p-cresol, thioglycollic acid, thiophenol.

69. The affinity matrix of claim 65, wherein R1 and R2 are selected from the group of amines of Table 1.

70. (canceled)

71. A process for the enrichment of low abundance proteins from proteinacious samples comprising carrying out affinity chromatography using as the general affinity matrix of claim 61.

72. (canceled)
